(19) United States
(12) Patent Application Publication
(10) Pub. No.: US 2010/0106051 A1
(43) Pub. Date: Apr. 29, 2010

(54) SINGLE LUMEN CATHETER WITH SEPARATE TUBES THEREIN

(75) Inventor: Donald E. Bobo, SR., Fountain Valley, CA (US)

Correspondence Address: INSKEEP INTELLECTUAL PROPERTY GROUP, INC, 2281 W. 190TH STREET, SUITE 200, TORRANCE, CA, 90504, US

(73) Assignee: InnerSpace, Inc.

(21) Appl. No.: 12/606,154

(22) Filed: Oct. 26, 2009

Related U.S. Application Data
(60) Provisional application No. 61/197,039, filed on Oct. 24, 2008.
(60) Provisional application No. 61/197,041, filed on Oct. 24, 2008.

(51) Int. Cl. A61M 25/00; A61B 5/00

(52) U.S. Cl. 600/561; 604/523

(57) ABSTRACT

A catheter is provided that includes multiple passages within a catheter body. These passages are arranged in a way so as to maximize the usable space within the catheter body. More specifically, multiple tubes are positioned within a central lumen of the catheter. Both the tubes and the central lumen are used as passages for various functionality of the catheter, such as communication of air or fluids. By sharing the free space within the central lumen of the catheter, the flow area of this passage is maximized as compared with simply using an extruded lumen for each passage. Hence, the catheter can allow the addition of more passages than similarly sized prior art catheters.

### RELATED APPLICATIONS

This application claims priority to U.S. Provisional Application Ser. No. 61/197,039 filed Oct. 24, 2008 entitled A Single Lumen Catheter with Separate Tubes Therein and U.S. Provisional Application Ser. No. 61/197,041 filed Oct. 24, 2008 entitled A Catheter with an Integrated Pressure Sensor all of which are hereby incorporated herein by reference.

### BACKGROUND OF THE INVENTION

During medical procedures, catheters are often inserted into various locations of a patient, such as vessels, ducts, and body cavities. Typically, these catheters are configured for treatment or diagnostic purposes such as pressure sensing, fluid delivery, stent delivery, and numerous other functions.

While some catheters are configured for a single purpose, such as only fluid delivery, other catheters are configured for multiple functions. Often these multi-function catheters require multiple lumens in their interior to allow for their various functions. For example, one lumen may be dedicated to an in-vivo air-based pressure sensor while another passage may be dedicated to delivery of fluids.

One drawback to such extruded multi-function catheters is that as additional lumens are added to a catheter, either the catheter diameter must be increased or the diameter of each lumen must be decreased, so as to accommodate the additional lumens. Often, increasing the diameter of the catheter can decrease the performance of the catheter. For example, catheters over a certain diameter may be difficult to insert or maneuver in particular areas of a body. Similarly, decreasing the lumen size in a catheter can decrease performance of the lumen-related functionality. For example, the rate of fluid delivery may be decreased or the accuracy of measurements from an air-based pressure sensor may be decreased.

Hence, the functionality of catheters is often limited to a relatively small number of features so as to balance these features with performance considerations.

### SUMMARY OF THE INVENTION

In a preferred embodiment according to the present invention, a catheter is provided that includes multiple passages within a catheter body. These passages are arranged in a way so as to maximize the usable space within the catheter body. More specifically, multiple thin-walled tubes are positioned within a central lumen of the catheter. Both the tubes and the central lumen are used as passages for various functionality of the catheter, such as communication of air or fluids. By passing thin-wall tubes through the free space within the central lumen of the catheter, the total flow area of the catheter is maximized as compared with an extrusion with multiple lumens as the total cross section area of the tubes is less than the extruded walls they replace. Hence, the catheter can allow the addition of more passages than similarly sized prior art catheters.

Preferably, at least one of the passages of the catheter terminates in a closed lumen on the distal end of the catheter. For example, at least one passage terminates in a sealed chamber of a pressure sensor composed of a flaccid tube. In this respect, some distal openings of the catheter are open to the outside environment at the outside of the distal end and at least one opening terminates with an enclosure, lumen or other non-open portion.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which:

FIG. 1A illustrates a side view of a catheter according to the present invention;

FIG. 1b illustrates a cross sectional view taken along lines a-a in FIG. 1A;

FIG. 2 illustrates an exploded side cross sectional view of the catheter of claim 1A;

FIG. 3 illustrates an exploded view of a pressure sensing assembly;

FIG. 4 illustrates a side view of a catheter with a pressure sensing assembly and a drainage assembly according to the present invention; and,

FIG. 5 illustrates a cross sectional view of a catheter body having four passages.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

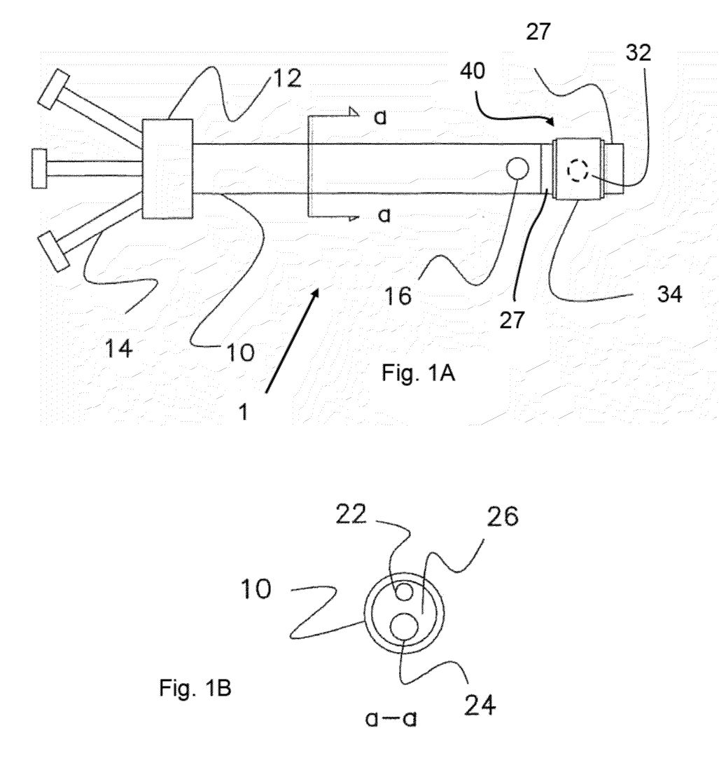

FIG. 1A illustrates a catheter 1 that includes multiple passages within a catheter body 10. These passages are arranged in a way so as to maximize the usable space within the catheter body. Hence, the catheter 1 can allow the addition of more passages than similarly sized prior art catheters.

It should be understood that this catheter design can be used for a variety of different purposes. In the present embodiment however, the catheter 1 is configured with one or more fluid delivery conduits (e.g., one or two) and an air conduit for pressure sensing. This design can be used for intra-cranial diagnostic and treatment.

Generally, the catheter 1 includes a plurality of flexible pigtail tubes 14 that are connected to a manifold 12 on the proximal end of the catheter body 10. A distal member 27 is secured, for example, by butt welding, to the distal end of the catheter body 10. Finally, a pressure sensing assembly 40 is located over a portion of the distal member 27.

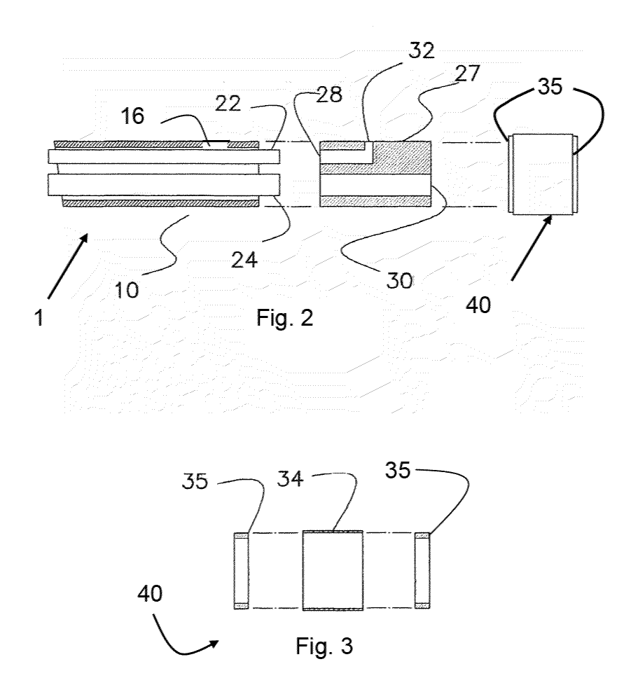

As best seen in FIGS. 1B and 2, three passages 22, 24 and 26 are located in the catheter body 10 and connect to a corresponding pigtail tube 14 through the manifold 12. More specifically, an air passage 11 and a fluid tube 24 are discrete tube members positioned within the central catheter lumen 26 of the catheter body 10. One pigtail tube 14 is coupled to the air passage 11, another pigtail tube 14 is coupled to the fluid tube 24, and another pigtail tube 14 is coupled to the central catheter lumen 26. In this respect, the free space of the central catheter lumen 26 acts as a passage or lumen for the third pigtail 14.

As best seen in FIG. 2, the distal member 27 includes distal fluid lumen 30, a distal air lumen 28 and a riser lumen 32. The fluid tube 24 is coupled to distal fluid lumen 30, which is open at the distal end of the catheter 1. In this respect, fluid can pass through the fluid tube 24, into the fluid lumen 30 and into the patient.

The air passage 22 is coupled to the distal air lumen 28 which exits the distal member 27 through riser lumen 32. Hence, air can pass through the air lumen 22, into the distal air lumen 28 and through the riser lumen 32.

In the present embodiment, the riser lumen 32 is not open to the interior of the patient. Instead, a pressure sensing assembly 40 is positioned over the riser lumen 32, as seen in FIG. 1A. As best seen in FIG. 3, the pressure sensing assembly 40 includes a flaccid tube 34 that is secured over the distal member 27 and riser lumen 32 by a clamping sleeve 35 on the proximal and distal ends of the flaccid tube 34 which are bonded in an airtight manner to the distal member 27.

Hence, as the pressure within the patient changes (e.g., intra-cranial pressure), the flaccid tube 34 moves, communicating this pressure change to the passage created by the riser lumen 32, the distal air lumen 28, the air tube 22 and to a pigtail tube 14. This arrangement allows a pressure sensor that is coupled this air passage to measure and display the pressure within the patient. Additional details about how such a pressure system operates can be found in U.S. Provisional Application No. 61/197,041, the contents of which are hereby incorporated by reference.

Finally, in the present embodiment, the distal member 27 does not provide a passage for the fluid passage created by the central catheter lumen 26 of the catheter body 10. Instead, a fluid aperture 16 in the catheter body 10 provides an exit for a second supply of fluid into the patient. While it is preferred that this fluid aperture 16 be located in the catheter body 16, it is also possible to create another lumen in the distal member 27, so long as the exit of this lumen is not covered or obstructed by the sensing assembly.

By utilizing the central catheter lumen 26 of the catheter body 10 instead of adding another tube within the catheter body 10, the fluid passage space for this fluid passage is maximized. Hence, the overall diameter of the catheter body 10 can be minimized without affecting desired performance.

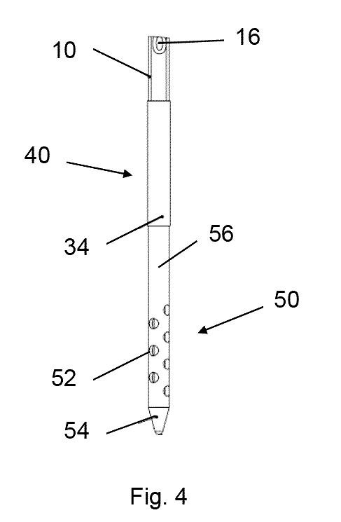

FIG. 4 illustrates a more specific example catheter according to the present invention that includes both a pressure assembly 40 and a drainage assembly 50. The drainage assembly 50 includes a cylindrical member 56 having an internal passage connected to the distal fluid lumen 30. A plurality of drainage apertures 52 connect the internal passage of the cylindrical member 56 with the environment around the catheter. A soft tip 54 reduces any contact-related trauma. Hence, this catheter example provides both drainage and pressure sensing to an area within a patient, such as within a brain, while maximizing the internal space within the catheter body 10.

While three passages or conduits have been described, it should be understood that fewer or more passages may also be included according to the present invention. For example, the central lumen of the catheter body may include 3 distinct tubes (4 passages total), 4 distinct tubes (5 passages total) and 5 distinct tubes (6 passages total).

### Example 1

In one example according to the present invention, a catheter can be created by first providing a single lumen catheter. Two thin-walled polyimide tubes are positioned down the central lumen of the catheter. The proximal end of the catheter and two tubes are bonded to a manifold having three passages, each of which connects to a pigtail tube. Two of the pigtail tubes connect to each of the two tubes while a third pigtail tube and manifold passage connect to the central lumen of the catheter.

An aperture is placed in the wall of the catheter near its distal end such that fluid can pass into one pigtail tube, around the outside for the remaining two polyimide tubes and into the central lumen and out the aperture. Preferably, the remaining area of the central lumen (the total central lumen area minus the area of the two polyimide tubes) is about half the total central lumen area. This ratio allows the catheter to substitute for a larger catheter with a larger conduit.

In one example, the inner diameter of the central catheter lumen 26 is about 0.056 inches. The outer diameter of the air tube 22 about 0.018 inches and the outer diameter of the fluid tube 24 is about 0.035 inches. The flow area of the central catheter lumen 26 is about half of the flow area without the tubes 22 and 24. The distal member is preferably composed of a low durometer material so as to present a soft distal tip and therefore reduce damage when advancing the catheter 1 within a patient.

It should be appreciated that the space savings possible with the previously described invention may become more valuable as additional conduits are implemented. For example, if a sensor such as a dialysis membrane is placed distal to the pressure sensing assembly 40, it may be necessary to have two additional conduits. The difference between the diameter of a catheter using the central catheter lumen 26 as opposed to all individual tubes increases as the number of conduits increases (e.g., since additional area becomes devoted to passage walls for each conduit).

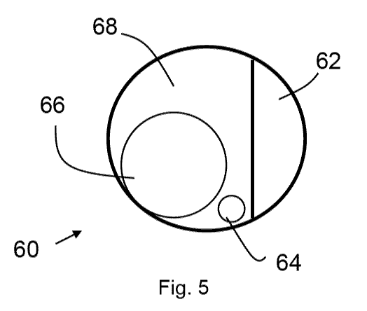

FIG. 5 illustrates a cross sectional view of another example catheter body 60 having 4 passages. More specifically, the catheter body 60 is divided into a main lumen 68 and a smaller lumen 62. Additionally, two passages 64 and 66 are located within the main lumen 68. Each of these passages 64 and 66 and lumens 62 and 68 are broken out into separate connection ports on a proximal end of the catheter body and to areas or ports on the distal end of the catheter body.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A catheter having multiple passages comprising:
a catheter body having an elongated shape, a distal end and a proximal end;
a lumen located within said catheter body and in communication with a first aperture near a distal end of said catheter body;
a first tube located in said lumen and in communication with a second aperture near said distal end of said catheter body; and,
a second tube located in said lumen and in communication with a third aperture near said distal end of said catheter body.

2. The catheter of claim 1, further comprising a manifold connected to said proximal end of said catheter body, a proximal end of said first tube and a proximal end of said second tube.

3. The catheter of claim 1, wherein said catheter body further comprises a distal member and wherein said second aperture and said third aperture are located on said distal member.

4. The catheter of claim 3, wherein said distal member seals a distal end of said lumen.

5. The catheter of claim 4, further comprising a pressure measurement assembly coupled to said distal member.

6. The catheter of claim 5, wherein said pressure measurement assembly is disposed over said second aperture.

7. The catheter of claim 6, wherein said pressure measurement assembly comprises a flaccid tube.

8. A medical device comprising:
a catheter body having an inner space forming a first passage;
a first tube disposed in said first passage and forming a second passage, sealed from said first passage;
a second tube disposed in said first passage and forming a third passage, sealed from said first passage and said second passage; and,
a manifold disposed on a proximal end of the catheter body and having a first manifold tube in communication with said first passage; a second manifold tube in communication with said second passage and a third manifold tube in communications with said third passage.

9. The medical device of claim 8, further comprising a first aperture located near a distal end of said catheter body and being in communication with said first passage; a second aperture located near a distal end of said catheter body and being in communication with said second passage; and a third aperture located near a distal end of said catheter body and being in communication with said third passage.

10. The medical device of claim 9, further comprising a flaccid tube disposed over said first aperture; said flaccid tube having a proximate end bonded proximal to said first aperture and a distal end bonded distal to said first aperture.

11. The medical device of claim 10, wherein said catheter body comprises a distal member disposed on a distal end of a proximal catheter body member; said distal member having a first lumen in communication with said first tube and said second aperture and a second lumen in communication with said second tube and said third aperture.

12. The medical device of claim 11, further comprising a pressure sensing assembly disposed over said first aperture.

13. The medical device of claim 12, wherein said pressure sensing assembly includes a flaccid tube having a distal end and a proximal end bonded to said distal member.

14. The medical device of claim 13, wherein said distal member is composed of a material having a lower durometer rating than said proximal catheter body member.

15. A multi-lumen catheter for use in a patient comprising:
an elongated catheter body forming a first passage, a second passage and a third passage;
said second passage and said third passage comprising a first tube and a second tube, respectively; and
said first tube and said second tube being fully disposed within said first passage.

16. The multi-lumen catheter of claim 15, further comprising a manifold disposed on a proximal end of said catheter body, said manifold connecting to a proximal end of said first tube, a proximal end of said second tube and a proximal end of said first passage.

17. The multi-lumen catheter of claim 16, further comprising a distal member disposed on a distal end of said catheter body and connecting to said first tube and said second tube.

18. The multi-lumen catheter of claim 17, wherein said distal member comprises a first distal lumen connected to said first tube and open to an exterior of said catheter body and a second distal lumen connected to said second tube and open to an exterior of said catheter body.

19. The multi-lumen catheter of claim 18, further comprising a flaccid tube sealed over an aperture in communication with said first distal lumen.

20. The multi-lumen catheter of claim 19, further comprising a second aperture disposed on a distal end of said catheter body and in communication with said first passage.
